(19) United States
(12) Patent Application Publication
(10) Pub. No.: US 2013/0284338 A1
(43) Pub. Date: Oct. 31, 2013

(54) SELF ASSEMBLY OF GRAPHENE MATERIALS

(71) Applicants: Xingcheng Xiao, Troy, MI (US); Zhongyi Liu, Troy, MI (US)

(72) Inventors: Xingcheng Xiao, Troy, MI (US); Zhongyi Liu, Troy, MI (US)

(73) Assignee: GM Global Technology Operations LLC., Detroit, MI (US)

(21) Appl. No.: 13/456,263

(22) Filed: Apr. 26, 2012

(51) Int. Cl. B32B 37/14; C07C 5/00; C01B 31/04; B82Y 40/00

(52) U.S. Cl. 156/60; 423/448; 585/400; 977/842

(57) ABSTRACT

Graphene and graphene-like materials may be formed by preparing a solution of a suitable polycyclic aromatic hydrocarbon (PAH) in a solvent that is immiscible with water (or other suitable underlying liquid). A suitably thin layer of the PAH solution is formed on the surface of a thin layer of water. The solvent is evaporated from the solution layer to form a film of PAH material organized in contiguous molecular discs. The organized PAH material may be further processed by careful removal or evaporation of the water layer to deposit the PAH residue on a desired surface. The PAH residue may then be heated to remove hydrogen atoms and form a carbon-enriched or wholly carbon, graphene structure.

### TECHNICAL FIELD

This disclosure is related to making graphene and graphene-like materials from polycyclic aromatic hydrocarbon molecules.

### BACKGROUND OF THE INVENTION

Graphene is a flat monolayer of sp²-bonded carbon atoms arranged in a two-dimensional hexagonal lattice. As such, graphene is also a basic building block of various graphitic materials, such as graphite. For example, crystalline flake graphite has a three-dimensional structure consisting of several stacked layers of graphene with interlayer bonding.

Graphene has potential for use in a wide variety of applications due to its large specific surface area, its excellent in-plane mechanical strength, and its high thermal and electrical conductivity. For example, graphene has potential for use in sensor, transistor, and integrated circuit applications. Graphene further has potential use as a negative electrode material in lithium ion batteries, a catalyst support, a nanofiller for nanocomposites, and a lubricant additive. Graphite does not possess these same desirable properties because most of graphene's unique properties are associated with its two-dimensional planar structure.

Known methods of producing graphene include mechanical and chemical exfoliation of graphite, chemical reduction of graphene oxide, epitaxial growth from silicon carbide (SiC) and single crystalline copper substrates, and chemical vapor deposition on metal surfaces. But graphene layers produced by these methods tend to form agglomerates or to restack to form graphite.

There remains a need for an effective, low-cost method of synthesizing two-dimensional planar graphene layers. And there is a particular need for such a method in which the formed graphene layer(s) may be easily stored or transferred for different applications so as to make it more available for its many uses.

### SUMMARY OF THE INVENTION

This disclosure presents methods of forming graphene and graphene-like materials using polycyclic aromatic hydrocarbons (PAH) as starting materials. Polycyclic aromatic hydrocarbons are large planar molecules of high carbon content. Examples of polycyclic aromatic hydrocarbons include naphthalene, anthracene, phenanthrene, naphthacene, pyrene, coronene, and hexabenzo-coronene. Many PAH compounds with large molecular structures are derived from naphthalene, coal tar and petroleum. Many of these large molecular PAH compounds have not been named but they are characterized in their chemical structures as follows.

These condensed nuclear hydrocarbon compounds are typically formed of six-carbon atom rings with shared sides. Some of the rings in each molecule can be described with three double bonds and other rings in the molecule have one or two sets of double bonds. The compounds contain hydrogen atoms attached at available valance sites on carbon atoms. Some of the polycyclic aromatic hydrocarbon compounds may have occasional methyl groups (—CH₃) attached to a ring portion of the molecule. Because of the condensed aromatic ring structure each PAH compound generally contains less than one hydrogen atom per carbon atom. And the molecules of the many PAH-type compounds tend to be planar.

In accordance with the methods of this disclosure, one or more selected polycyclic aromatic hydrocarbon compounds are dissolved in an organic solvent that is lighter than water and immiscible with water. Toluene and a xylene (ortho-xylene, meta-xylene, or para-xylene, or mixtures) are examples of organic solvents that are useful in the practice of this graphene preparation method. A quantity of the bulk PAH compound material is dissolved in the organic solvent and the PAH solution is carefully dropped or distributed or otherwise carefully placed as a thin layer on the surface of a shallow layer of water. The shallow layer of water may be contained in a shallow pan, or other suitable container, as a processing medium. The container is used to confine the water layer and to present the water surface in a two-dimensional shaped area for the desired graphene sheet material to be formed. After the PAH solution has been prepared, this setup of the contained layers of water and PAH solution may generally be assembled at an ambient temperature (e.g., 20-25° C.) without heating of the constituent materials.

As will be seen, the layer of water (or other suitable liquid) serves as a temporary fluid foundation for the PAH solution which now floats passively on the water surface. The organic solvent is carefully evaporated from the water surface to gradually leave an organized layer of molecules of the PAH compound(s) on the surface of the water. The solvent may be evaporated into a suitable reduced pressure atmosphere from which it may be recovered. But, as the solvent evaporates, the PAH material with its relatively flat molecules is organized into a very thin layer of ringed carbon material on the quiescent surface of the water. It is believed that pi orbital bonds in the PAH compound(s) and the hydrogen bonds in the water molecules of the water surface, possibly together with the surface tension of the water, cooperate in organizing the PAH molecules into a precursor layer of graphene-like material floating on the water surface. At first, free disks of PAH material may be formed with the evaporation of the organic solvent. As the evaporation of the PAH solvent nears completion, the organization of the PAH molecules increases with attraction of peripheral hydrogen bonds at the edges of the forming discs of carbon-rich compounds. The thickness of this layer is typically of the order of a few Angstroms.

The water is evaporated so as to avoid damage or mis-shaping of the generally one layer to a few layers of PAH material. Some suitable moderate heating may be provided, such as from the underside of the contained shallow water layer. And vaporization of the water may be promoted with the establishment of a reduced pressure atmosphere over the surface of the system. The initial depth of the water may have been only a couple of millimeters or so. With the complete removal of the water, a dry film is now deposited on one or more solid surfaces originally selected to underlie and support the shallow water layer.

The solid surface on which the very thin layer of PAH compound material comes to rest may be of any suitable material for supporting the flat molecular layers. However, in many practices of this process it may be preferred to use copper or silicon carbide as the solid layer for deposition of the graphene precursor material and for removal of hydrogen from it. As stated the solid surface may comprise separate sections, discs, wafers, or other shapes or members for separate recovery and treatment of the PAH compound material.

There may be some practices of the process in which the thin layer of PAH compound with high carbon content is suitable for an intended application. It is graphene-like in character but typically contains more hydrogen than graphene. Accordingly, in many practices it will be preferred to heat the PAH compound, for example, resting on its solid platform, to remove hydrogen from the molecules of the polyaromatic hydrocarbon compound. The material may be heated through its solid support layer with the upper surface exposed to a vacuum or other reduced pressure environment. The surface of the PAH compound may be protected with, for example, an argon atmosphere. And the inert gas atmosphere may be heated and flowing to carry off hydrogen driven from the compound. At the completion of the heating a single layer, or relatively few layers, of a two dimensional network of interconnected rings of carbon atoms is obtained. The graphene structure may be handled and used as graphene structures prepared by other practices, such as exfoliation practices or bottom-up growth practices.

In accordance with practices of this invention, the term “graphene” is used to refer to a substantially flat monolayer of sp²-bonded carbon atoms arranged in a two-dimensional hexagonal lattice. Graphene contains few if any hydrogen atoms. The term “graphene-like” refers to like arrangement of carbon atom rings which still contain some hydrogen atoms that prevent the formation of wholly flat monolayer of sp²-bonded carbon atoms arranged in a two-dimensional hexagonal lattice.

Thus, a simple and efficient method for producing graphene and graphene-like structures from PAH compound precursors has been disclosed. Other objects and advantages of this invention will be understood from further descriptions and illustrations of the invention, which follow in this specification.

### BRIEF DESCRIPTION OF THE DRAWINGS

FIG. 1 is an oblique view of the upper surface of a rectangular pan for holding, from the bottom of the pan upwardly, a solid base layer, a shallow layer of water, and a shallow layer of a solution of a polycyclic aromatic hydrocarbon compound floating on the water layer. This arrangement serves to illustrate a practice of the method of forming an organized layer of graphene or graphene-like material.

FIG. 2A is an enlarged broken-out fragmentary view of a small portion of FIG. 1, taken at location “2” of FIG. 1. FIG. 2 illustrates, in sectional view, the solid base layer, a shallow layer of water, and the shallow layer of the solution of the polycyclic aromatic hydrocarbon compound floating on the water layer at the beginning of the practice of a method for synthesizing a layer of graphene from the PAH.

FIG. 2B is an enlarged fragmentary view, similar to FIG. 2A, illustrating self-organized PAH compound floating on the shallow water layer after the solvent for the PAH has been evaporated.

FIG. 2C is an enlarged fragmentary view, similar to FIG. 2B, showing the self-organized PAH molecular film resting on the solid base layer after the water has been evaporated.

FIG. 3 is an oblique view of the solid base layer removed from its pan and carrying the PAH preparatory to graphitization for the formation of graphene.

FIG. 4 is an enlarged optical image of a self-assembled PAH graphene precursor layer floating on water as illustrated schematically in FIG. 2B.

FIG. 5 is a high resolution transmission electron microscopic view (HRTEM) of a self-assembled, PAH graphene precursor layer from which the underlying water has been removed. The small square image in the lower right corner of FIG. 5 is an enlarged view of a section of the precursor layer as indicated by the arrow.

FIG. 6 is a high resolution transmission electron microscopic view (HRTEM) of a self-assembled, PAH graphene precursor layer after removal of the water.

FIG. 7 is an illustration of chemical formulas of unnamed PAH compounds, derived respectively, from coal-tar, petroleum, and naphthalene, and suitable for use in forming graphene in accordance with practices of this invention.

### DESCRIPTION OF PREFERRED EMBODIMENTS

As described above, the preparation of graphene and graphene-like materials in accordance with practices of this disclosure involve the processing of a thin layer of a solution of a PAH compound floated on a thin layer of water. The solvent from the PAH solution is to be evaporated, and, in many embodiments, the water is subsequently evaporated. It is recognized that the handling of the two liquid layers maybe managed in many ways, including batch-processing ways and continuous-processing ways. For purposes of less complicated illustration, a practice of the invention will be illustrated using a simple batch processing container for the liquid layers.

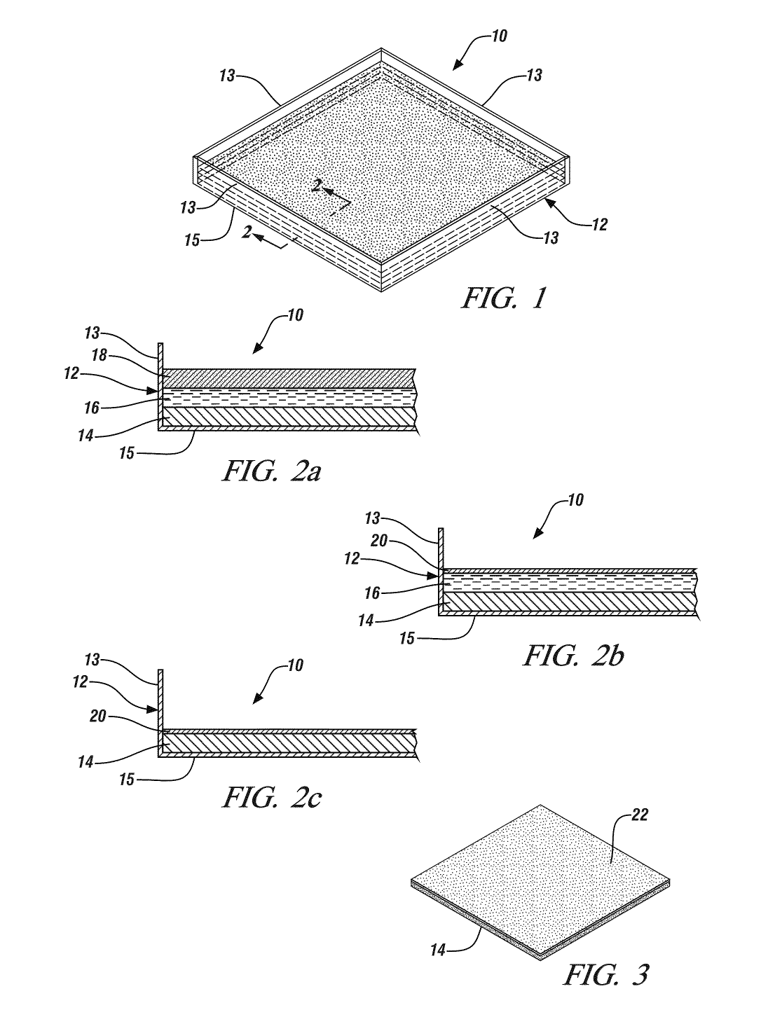

In FIG. 1, a processing system or arrangement 10 is shown using a rectangular shaped processing pan 12 with four vertical sides 13 and a flat base 15. The sides 13 may be attached to the base 15 of the pan 12 so that they can be dropped or folded outwardly toward or into the plane of the base 15 of the pan 12. For example, the sides 13 may be hinged (not illustrated) to the base 15 of the pan 12. The shape of the pan 12 (or other processing equipment) is determined in part by the desired planar shape of the molecular layer of graphene or graphene-like material to be made. In this example, a rectangular shape of several inches on each side is used as an example of the processing. Pan 12 contains two liquid layers which are better illustrated and described in FIG. 2A which is a fractional and enlarged cross-sectional view taken at location 2-2 of FIG. 1.

FIG. 2A is an enlarged fragmentary view of the vertical arrangement of the system 10 of FIG. 1 as it is contained in pan 12. In FIG. 2A, a relatively thin, flat, solid plate member 14 is placed on the bottom 15 of pan 12. The composition and purpose of the thin, flat, solid member 14 will be described as it is further involved in the preparation of graphene or graphene-like material. While a single solid member 14 is illustrated, two or more smaller solid members, of like or different shapes, may be used, for example, to recover separate portions of graphene or graphene-like material produced by the subject process. Pan 12 contains a layer of water 16 on solid plate member 14 and a layer of PAH compound solution 18 floating on water layer 16. The layer of water 16 may be a few millimeters in depth and during the practice of the method it is maintained sufficiently quiescent so that the layer of the PAH solution is also still.

In many embodiments of the invention, the pan 12 and its contents 14, 16, 18 may have been assembled without any heating. The initial PAH solution may be formed with or without heating, depending on the specific combination of the PAH solute material and the water-immiscible solvent. An aromatic organic solvent such toluene or xylene is often a preferred water-immiscible solvent for the PAH material. Once the PAH solution has been prepared it is added as drops or a slow stream to form layer 18 of the solution on the layer of water 16, previously placed in pan 12. The PAH material solution 18 is prepared so as to provide a layer of PAH molecules on the surface of the water when the solvent is removed.

The initially unheated pan 12 with water layer 16 and PAH solution 18 may be placed in a suitable closable, oven-like, chamber (not illustrated) for evaporation of the liquid solvent material of the PAH solution 18. The pressure of the air in the chamber may be reduced at a rate to promote evaporation of the solvent from the PAH solution, preferably without causing bubbling or other disturbances in solution layer 18 that would disturb the self-organization of the molecules of the PAH material in the shrinking body layer of the solution layer 18. Or the air may be replaced with a gas, such as nitrogen or argon that is more inert with respect to the solvent being evaporated. And moderate heat of the solution layer may be applied to the solution layer 18 to enhance removal of the solvent. Where practical the solvent is captured and condensed for reuse.

FIG. 2B is a schematic illustration of the system 10 after the solvent has been removed from the evaporation chamber (not illustrated). The residual, substantially solvent-free PAH material 20 now remains floating on the surface of water layer 16. The schematic illustration of layer 20 in FIG. 2B is enlarged and intended to show the stage of the process and the formation of the PAH layer by removal of the solvent. The PAH layer 20 is actually very thin, only a few molecules thick, and it represents graphene-like material floating on the much smaller molecules of the water layer 16. The water surface tension and the interaction of the relatively large polycyclic aromatic hydrocarbon molecules lead to the self assembly of the molecules of PAH as layers of graphene-like material. FIG. 4 is an enlarged optical image of discs and bodies of assembled PAH molecules on the water surface. As seen in the image, the main disc spans areal dimensions of millimeter scale while the graphene-like material is only a few molecules thick.

The graphene-like material 20 at the processing stage depicted in FIG. 2B will have some utility as is. For example, the material 20 may be collected for use in nano-fluids to improve thermal conductivity. But in many embodiments of the invention it is desired to separate the graphene-like material 20 from the water layer 16 on which it is floating in pan 12.

Pan 12 and its remaining contents, solid base layer 14, water layer 16, and graphene-like material 20 may still be in the chamber in which the solvent was evaporated from the original system 10. An inert gas such as argon may now be slowly flowed through the processing chamber, under reduced pressure, to begin careful removal of the water layer 16. The inert gas may be heated as found necessary, and moderate heat may be applied through the bottom 15 of pan 12. The heating and flow of inert gas is managed to remove the water layer 16 and to deposit the layer of graphene-like material 20 on the solid base plate 14. As the small water molecules are being released through the larger PAH molecular structure, the sides 13 of pan 12 may also be slowly and timely dropped to permit removal of the water at the sides of the assembly. When the water layer 16 has been fully removed, the graphene like material 20 now rests on base layer 14 as illustrated in FIG. 2C.

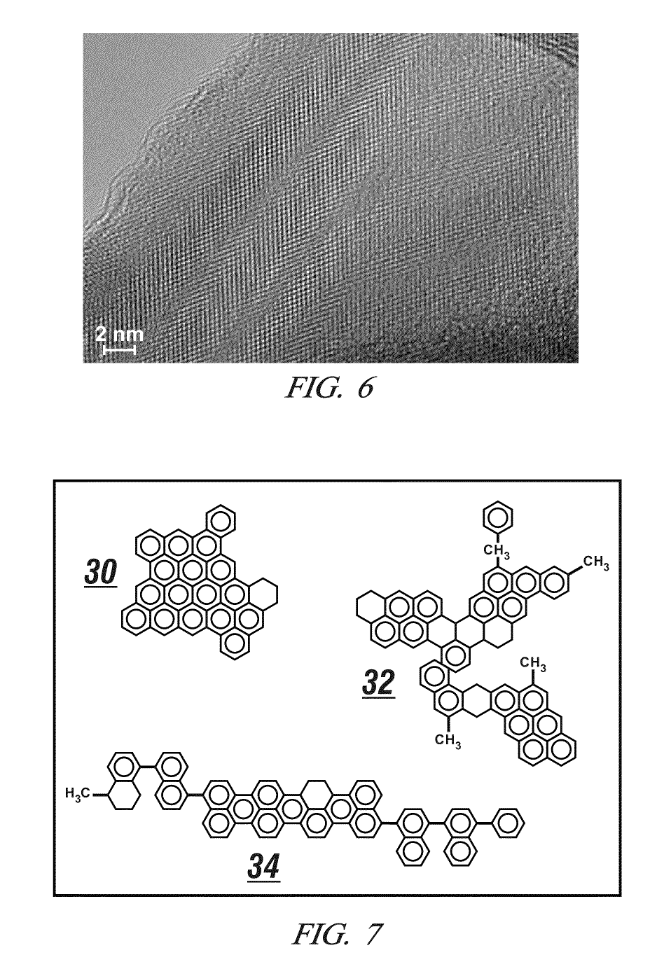

At this processing stage the pan 12 may be removed from the processing chamber with base layer 14 with its coating of graphene-like material 20. This residue of material 20 in FIG. 2C may be considered as graphene-like material because it still contains residual hydrogen. FIG. 5 is a high resolution transmission electron microscope (HRTEM) image of graphene-like material in which the PAH material floating on water taken as a TEM sample and placed on the TEM grid as a “fishing net” to separate the graphene-like material from the water. The HRTEM image of FIG. 5 shows the self-organized layer structure of the graphene-like material. The further enlarged image in the box in the lower right corner of FIG. 5 illustrates a small square portion of the material (indicated by the arrow) that is about one nanometer on a side. And FIG. 6 presents a further and enlarged image of the self-assembled, graphene-like layer. This layer of graphene-like material was formed from the naphthalene-derived compound illustrated in the structural formula 34 of FIG. 7.

As stated above, the graphene-like material 20 on a solid plate 14 in FIG. 2C is in the form of a very thin layer of self-organized molecular material precipitated from solution by evaporation of its solvent. The material was initially deposited on water, but the water has been removed at the process step of FIG. 2C. This material is considered as graphene-like because of its residual content of hydrogen which is not present in graphene. So the graphene like material as deoposited on its solid base may now be further heat treated to remove its residual content of hydrogen.

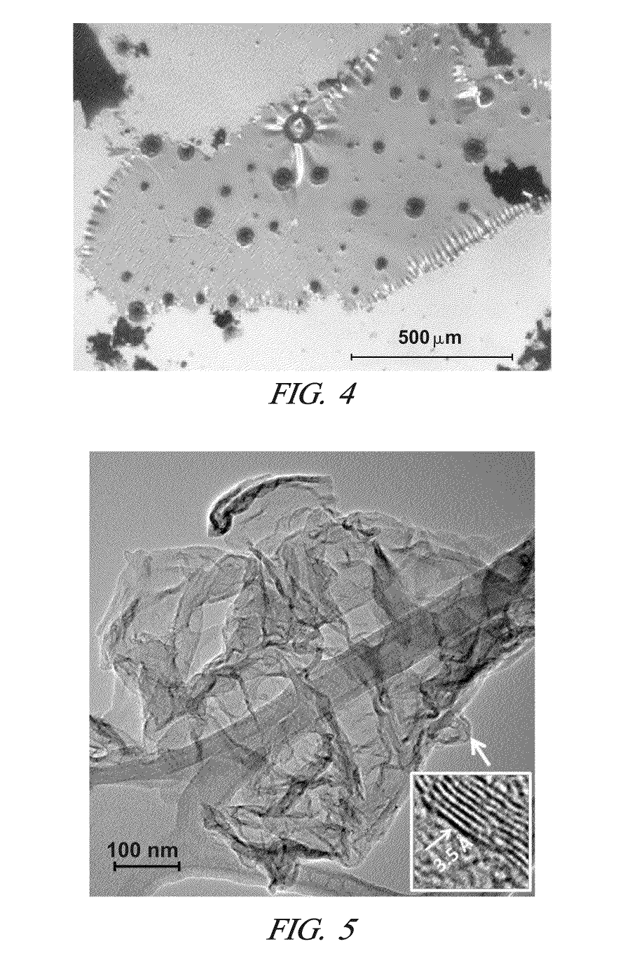

As schematically illustrated in FIG. 3, the graphene-like material 20 on it planar solid base 14 may be further heat treated under reduced pressure to remove hydrogen from the material. This heat treatment may be conducted under flowing hydrogen at reduced pressure to yield graphene 22 on a supporting substrate 14.

As stated above, FIG. 7 is an illustration of structural chemical formulas of unnamed PAH compounds, derived respectively, from coal-tar (30), petroleum (32), and naphthalene (34), and suitable for use in forming graphene in accordance with practices of this invention.

Thus, we have provided practices for forming graphene-like materials and graphene. The graphene-like material is initially formed as a self-assembled film on a layer of water. With further processing the underlying water layer is removed and the self-assembled film of graphene-like material may be used on a solid plate, disc, wafer, or the like off copper, silicon carbide, or other desired substrate material. With still further processing, residual hydrogen may be removed from the precursor self-organized material layer to form substantially pure graphene.

Of course the precursor materials may be lifted from a water layer or solid plate for use in an application as desired.

In the above described practice of the invention, water was used as the supporting liquid layer for the initial solution of the PAH material. Water is preferred because of its obvious availability and utility. It is an inexpensive material that is immiscible with many suitable organic solvents for PAH materials, that has a higher specific gravity that most such solutions, and water is liquid at most ambient temperatures. However, if preferred, a suitable different liquid may used as the supporting liquid for the PAH solution.

Further, in the above illustration of a practice of the invention, a simple rectangular pan was used in the process. But the process may be practiced as a batch-type process in other container shapes for the initial assembly of the supporting plate and the liquid layers. A single solid base layer may be used or many separate discs, wafers, or other solid substrates may be used to recover separate portions of the self-organized graphene or graphene-like material. And the process may be practiced in a continuous process where the liquid layers are initially assembled and the layers progressively removed as the materials are continually (or semi-continually) advanced using a supporting medium.

While the disclosure has been presented to illustrate practices of the invention, the illustrations are not presented as limitations of the invention.

1. A method of forming graphene-like material or graphene comprising:
forming a liquid solution of a polycyclic aromatic hydrocarbon in a solvent such that the solution will float on the surface of a layer of water without substantial dissolution or extraction of the polycyclic aromatic hydrocarbon into the water;
forming a layer of the solution of the polycyclic aromatic hydrocarbon on the surface of a layer of water, the layer of water and the overlying layer of the solution being contained to form a surface area for a desired planar configuration for the solution of polycyclic aromatic hydrocarbon; and
evaporating the solvent from the layer of solution of the polycyclic aromatic hydrocarbon to form a residual layer of polycyclic aromatic hydrocarbon, graphene-like material on the layer of water, the residual layer of polycyclic hydrocarbon being in the form of a substantially molecular film of organized molecules of polycyclic aromatic hydrocarbon that are substantially compliant with the surface of the water layer, the film having a thickness of less than a nanometer.

2. A method as recited in claim 1 and further comprising;
containing the layer of water over a base of solid material for receiving the film of organized molecules of polycyclic aromatic hydrocarbon; and
evaporating the water layer to deposit the film of organized molecules of polycyclic aromatic hydrocarbon, graphene-like material onto the base of solid material.

3. A method as recited in claim 2 and further comprising;
heating the film of organized molecules of polycyclic aromatic hydrocarbon under vacuum or an inert atmosphere to reduce the content of hydrogen atoms in the material to leave a carbon-enriched residue on the base of solid material, the carbon-enriched residue being characterized by one or more two-dimensional layers of connected six-member rings of carbon atoms in nature of graphene.

4. A method as recited in claim 1 in which the solvent is an aromatic organic compound selected from the group consisting of toluene and xylene.

5. A method as recited in claim 2 in which the base of solid material comprises silicon carbide.

6. A method as recited in claim 2 in which the base of solid material comprises copper.

7. A method of forming graphene or graphene-like material comprising:
forming a liquid solution of a polycyclic aromatic hydrocarbon in an aromatic organic compound solvent such that the solution is immiscible with water;
forming a layer of the water-immiscible solution of the polycyclic aromatic hydrocarbon on the surface of a layer of water of a depth up to a few millimeters at an ambient temperature and without heating, the layer of water being contained on a base of solid material and confined on its sides to form a surface area for a desired planar configuration of the graphene or graphene-like material;
reducing the atmospheric pressure over the solution layer and evaporating the solvent from the layer of solution of the polycyclic aromatic hydrocarbon to form a residual layer of polycyclic aromatic hydrocarbon on the layer of water, the residual layer of polycyclic hydrocarbon being in the form of a film of (i) a continuous body or (ii) discontinuous bodies of organized molecules of polycyclic aromatic hydrocarbon that are substantially compliant with the surface of the water layer, the film having a thickness of less than a few nanometers;
evaporating the water layer to deposit the film of organized molecules of polycyclic aromatic hydrocarbon onto the base of solid material; and
heating the film of organized molecules of polycyclic aromatic hydrocarbon in a vacuum or inert atmosphere to reduce the content of hydrogen atoms in the material to leave a carbon-enriched residue on the solid material, the carbon-enriched residue being characterized by one or more two-dimensional layers of connected six-member rings of carbon atoms, in nature of graphene.

8. A method of forming graphene or graphene-like material as recited in claim 7 in which the organic aromatic compound solvent is a compound selected from the group consisting of toluene and xylene.

9. A method as recited in claim 7 in which the base of solid material comprises silicon carbide.

10. A method as recited in claim 7 in which the solid material comprises single crystalline copper.

11. A method of forming graphene-like material or graphene comprising:
forming a liquid solution of a polycyclic aromatic hydrocarbon in a first solvent;
forming a layer of the solution of polycyclic aromatic hydrocarbon on an underlying supporting layer of a liquid that is immiscible with the liquid solution;
evaporating the first solvent to form a substantially single molecular layer of the polycyclic aromatic hydrocarbon on the liquid supporting layer;
removing the liquid supporting layer and depositing the molecular layer of polycyclic aromatic hydrocarbon on a substrate as a graphene-like material.

12. A method as recited in claim 11 in which the graphene-like material is heated on the substrate to remove hydrogen from the graphene-like material and to form graphene.

13. A method of forming graphene or graphene-like material as recited in claim 11 in which the first solvent is a compound selected from the group consisting of toluene and xylene.

14. A method as recited in claim 11 in which the substrate comprises silicon carbide.

15. A method as recited in claim 11 in which the substrate comprises single crystalline copper.

16. A method as recited in claim 1 in which the polycyclic aromatic hydrocarbon is derived from a material selected from the group consisting of naphthalene, coal tar, and petroleum.

17. A method as recited in claim 7 in which the polycyclic aromatic hydrocarbon is derived from a material selected from the group consisting of naphthalene, coal tar, and petroleum.

18. A method as recited in claim 11 in which the polycyclic aromatic hydrocarbon is derived from a material selected from the group consisting of naphthalene, coal tar, and petroleum.

19. A method as recited in claim 1 in which the layer of the solution of polycyclic aromatic hydrocarbon and underlying layer of water are contained in a pan defining the desired area of the liquids for the formation of the graphene-like material or graphene, and the liquids are contained over a single-piece base of solid material for receiving the entire residue of graphene-like material or graphene after removal of the solvent and water.

20. A method as recited in claim 1 in which the layer of the solution of polycyclic aromatic hydrocarbon and underlying layer of water are contained in a pan defining the desired area of the liquids for the formation of the graphene-like material or graphene, and the liquids are contained over a multi-piece base of solid material such that individual pieces of the multi-piece base carry portions of the graphene-like material or graphene after removal of the solvent and water.
